(19) United States
(12) Patent Application Publication
(10) Pub. No.: US 2026/0175051 A1
(43) Pub. Date: Jun. 25, 2026

(54) Respiratory stabilization device, radiation delivery system, and respiratory stabilization method

(71) Applicant: Atsuya Takeda, Saitama (JP)

(72) Inventor: Atsuya Takeda, Saitama (JP)

(21) Appl. No.: 19/420,650

(22) Filed: Dec. 15, 2025

(30) Foreign Application Priority Data
Dec. 19, 2024 (JP) 2024223922

(51) Int. Cl. A61N 5/10

(52) U.S. Cl. CPC A61N 5/107; A61N 2005/1074; A61N 2005/1092

(57) ABSTRACT

The respiratory stabilization device includes an electrical stimulator attached to the abdomen of a user and applying electrical stimulation to the abdomen, a control block that controls the current supplied to the electrical stimulator, and an input block that can communicate with the control block and inputs respiratory cycle information, which is the user's respiratory cycle acquired in advance, to the control block. The control block is capable of adjusting a first period during which current is supplied to the electrical stimulator and a second period during which the current supply to the electrical stimulator is stopped, based on the respiratory cycle information.

### TECHNICAL FIELD

The present invention relates to a respiratory stabilization device, a radiation delivery system, and a respiratory stabilization method.

### BACKGROUND ART

Radiation delivery devices are widely used to deliver radiation to a lesion such as a tumor in a body. When the lesion is located in the chest or abdomen, the position of the lesion moves as the person breathes. Therefore, in order to prevent radiation from being irradiated to healthy tissues other than the lesion, it is necessary to stabilize the cycle and the amplitude of the respiratory movement.

As a means for stabilizing breathing patterns, for example, WO2023120232 (A1) discloses a breathing teaching device that includes an electrical stimulator that applies low-frequency stimulation to the abdomen and a control unit that controls the electrical stimulator to guide the person to perform stable breathing. The optimal breathing cycle for each user is different, and since the breathing teaching device described above cannot change the cycle at which low-frequency stimulation is applied to the abdomen for each person, it is difficult to stabilize the breathing cycle at an optimal cycle for each person or user.

### SUMMARY OF THE INVENTION

An object of one aspect of the present invention is to provide a respiratory stabilization device, a radiation delivery system, and a respiratory stabilization method that can stabilize the respiratory cycle at an optimal cycle for each user.

A respiratory stabilization device according to one embodiment of the present invention includes an electrical stimulator attached to the abdomen of a user and configured to apply electrical stimulation to the abdomen, a control unit for controlling a current supplied to the electrical stimulator, and an input unit capable of communicating with the control unit and inputting respiratory cycle information representing the user's respiratory cycle, the input unit being configured to adjust a first period during which the current is supplied to the electrical stimulator and a second period during which the supply of the current to the electrical stimulator is stopped, based on the respiratory cycle information.

One aspect of the radiation delivery system of the present invention includes the respiratory stabilization device described above and a radiation delivery unit that delivers radiation to a lesion in the chest or abdomen of the user, and the radiation delivery unit can adjust the radiation direction or period based on respiratory cycle information.

One aspect of the respiratory stabilization method of the present invention is a respiratory stabilization method using a respiratory stabilization device including an electrical stimulator attached to the abdomen of a user and applying electrical stimulation to the abdomen, a control unit controlling a current supplied to the electrical stimulator, and an input unit capable of communicating with the control unit, the method including: an input step of inputting respiratory cycle information representing the user's respiratory cycle acquired in advance to the control unit by the input unit; and a current supply step of supplying the current to the electrical stimulator by the control unit, wherein in the current supply step, the control unit is capable of adjusting a first period during which the current is supplied to the electrical stimulator and a second period during which the supply of the current to the electrical stimulator is stopped based on the respiratory cycle information.

According to one aspect of the present invention, a respiratory stabilization device, a radiation delivery system, and a respiratory stabilization method stabilize the respiratory cycle for each user at an optimal level.

### BRIEF DESCRIPTIONS OF DRAWINGS

FIG. 1 is a schematic block diagram showing a radiation delivery system according to an embodiment.

FIG. 2 is a perspective view showing a radiation delivery system according to an embodiment.

FIG. 3 is a schematic diagram showing an example of an attachment position of an electrical stimulator according to an embodiment.

FIG. 4 is a diagram showing an example of a current waveform according to the embodiment.

FIG. 5 is a flowchart illustrating a respiratory stabilization method according to an embodiment.

### REFERENCE NUMERALS AND SYMBOLS IN THE DRAWINGS

10 Radiation delivery system
11 Radiation delivery device
12 Gantry
13 Radiation control unit
15 Radiation head
17 Radiation delivery unit
18 Couch
19 Measurement block
19a Breath sound measurement unit
19c Displacement measurement unit
19e Measurement control unit
30 Respiratory stabilization device
31 Input block
31a Input unit
31c Breath sound sensor
31d Flow rate sensor
31e Displacement sensor
31g Micro controller unit
34 Control block
35 Control unit
36 Current supply unit
38 Electrical stimulator
39 Cable
41 Display unit
42 Sound unit
45 Stimulation interrupt unit
Ib Respiratory cycle information
Ic Current
In Aimed current
Is Supplied current
P User
R Radiation
Rc Operation room
Rt Treatment room
S01 Input step
S02 Current supply step
S03 Visual coaching step
S04 Audio coaching step
Ss Instruction sound
Ss1 First instruction sound
Ss2 Second instruction sound
St Stop signal
T1 First period
T2 Second period
Tb Respiratory cycle
Td Exhalation period
Ti Inhalation period
Vs Coaching image
Vs1 First coaching image
Vs2 Second coaching image
W Medical professional

### DETAILED DESCRIPTION OF THE INVENTION

Hereinafter, a respiratory stabilization device, a radiation delivery system, and a respiratory stabilization method according to embodiments of the present invention will be described with reference to the drawings. Note that the scope of the present invention is not limited to the following embodiments, and can be modified as desired within the scope of the technical concept of the present invention. In addition, in the following drawings, the scale and number of components may differ from the actual structure in order to make each component easier to understand.

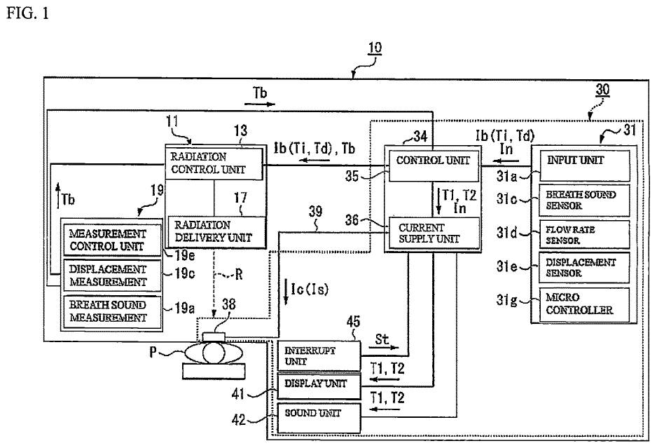

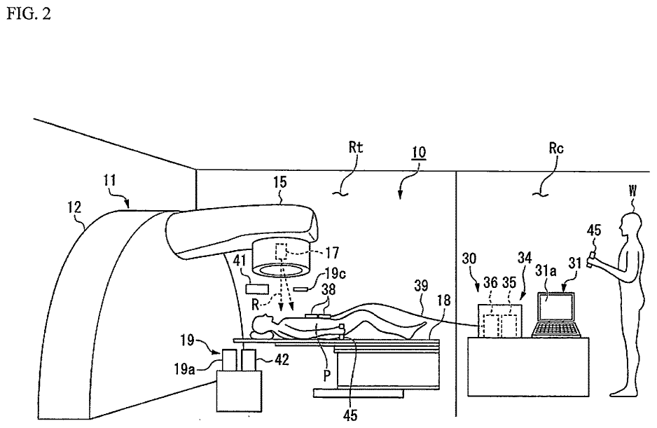

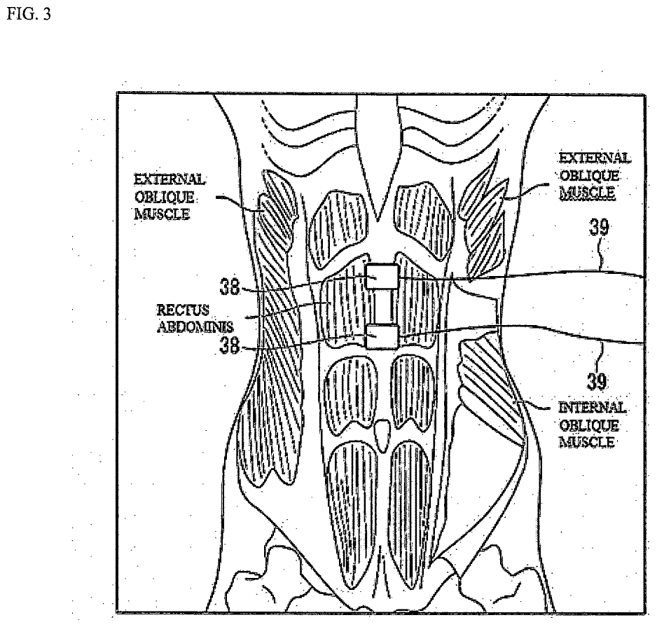

FIG. 1 is a schematic block diagram showing a radiation delivery system 10 of the present embodiment. FIG. 2 is a perspective view showing the radiation delivery system 10 of the present embodiment. FIG. 3 is a schematic diagram showing an example of the attachment position of an electrical stimulator 38 of the present embodiment. The radiation delivery system 10 of the present embodiment shown in FIGS. 1 and 2 is a medical device that delivers radiation to a lesion such as a tumor of a user P while stabilizing the respiratory cycle at an optimal cycle for each user P. As shown in FIG. 1, the radiation delivery system 10 of the present embodiment includes a radiation delivery device 11 and a respiratory stabilization device 30.

As shown in FIG. 2, the radiation delivery device 11 includes a gantry 12, a radiation head 15, a radiation delivery unit 17 and the measurement unit 19. As shown in FIG. 1, the radiation delivery device 11 includes a radiation control unit 13. The radiation delivery device 11 does not necessarily have to include the measurement unit 19. The gantry 12 and the radiation head 15 may be integrally configured. The radiation head 15 may also be attached to the tip of a robot arm (not shown).

The measurement block 19 measures the respiratory cycle Tb of the user P. In the present embodiment, the measurement block 19 has a breath sound measurement unit 19a and a displacement measurement unit 19c. The measurement block 19 does not necessarily have to have either the breath sound measurement unit 19a or the displacement measurement unit 19c. As shown in FIG. 1, the measurement block 19 has a measurement control unit 19e. Note that in the present embodiment, the “respiratory cycle Tb” refers to the “respiratory cycle of the user P when the lesion is treated by irradiating the user P with radiation R using the radiation delivery device 11.” The respiratory cycle Tb includes both an inhalation period during which the user P inhales air and an exhalation period during which the user P exhales air when the lesion is treated by irradiating the user P with radiation R.

The breath sound measurement unit 19a shown in FIG. 2 measures the breath sounds of the user P. More specifically, the breath sound measurement unit 19a measures the sounds when the user P inhales air and when the user P exhales air. In this way, the breath sound measurement unit 19a measures the breathing cycle Tb of the user P. In this embodiment, the breath sound measurement unit 19a is, for example, a microphone. The breath sound measurement unit 19a is capable of communicating with the measurement control unit 19e. The breath sound measurement unit 19a converts the breath sounds of the user P into electrical signals and transmits them to the measurement control unit 19e. The breath sound measurement unit 19a are placed near the face of the user P.

The displacement measuring unit 19c measures the displacement of the abdomen of the user P. More specifically, the displacement measuring unit 19c measures the displacement of the abdomen due to abdominal expansion when the user P inhales air and abdominal contraction when the user P exhales air. In this way, the displacement measuring unit 19c measures the user's respiratory cycle Tb. In this embodiment, the displacement measuring unit 19c is, for example, a laser displacement meter that irradiates the abdomen of the user P with infrared light and measures the position and displacement of the abdomen from the reflected light. The displacement measuring unit 19c is capable of communicating with the measurement control unit 19e. The displacement measurement unit 19c converts the displacement of the abdomen of the user P into an electrical signal and transmits it to the measurement control unit 19e. Note that the configuration of the displacement measurement unit 19c is not limited to this embodiment, and the displacement measurement unit 19c may be, for example, other measurement devices such as an acceleration sensor.

The unit 31g is a microprocessor such as an MCU (Micro Controller Unit). The measurement control unit 19e is capable of communicating with each of the breath sound measurement unit 19a, the displacement measurement unit 19c, and the radiation control unit 13. The measurement control unit 19e may be capable of communicating with each of the breath sound measurement unit 19a, the displacement measurement unit 19c, and the radiation control unit 13 via wired communication means such as a cable, or via wireless communication means such as a wireless LAN.

The measurement control unit 19e derives the respiratory cycle Tb of the user P based on the electrical signals transmitted from the breath sound measurement unit 19a and the displacement measurement unit 19c. The respiratory cycle Tb includes a period when the user P exhales air and a period when the user P inhales air. The measurement control unit 19e transmits the derived respiratory cycle Tb of the user P to the radiation control unit 13. Note that the measurement block 19 does not need to include the measurement control unit 19e. In this case, both the breath sound measurement unit 19a and the displacement measurement unit 19c transmit electrical signals to the radiation control unit 13, and the radiation control unit 13 derives the respiratory cycle Tb of the user P. Furthermore, in this embodiment, the measurement control unit 19e is capable of direct communication with the control unit 35. This allows the measurement control unit 19e to transmit the respiratory cycle Tb of the user P to the control unit 35. Note that the measurement control unit 19e does not need to be capable of direct communication with the control unit 35.

As shown in FIG. 2, the radiation delivery device 11 is installed in a treatment room Rt. The gantry 12 is installed, for example, on a wall of the treatment room Rt. The gantry 12 rotatably supports a radiation head 15. The radiation head 15 is a housing that extends horizontally. A radiation delivery unit 17 is built at the end of the radiation head 15.

The Radiation delivery unit 17 irradiates radiation R to the lesion of a user P lying on a couch 18. The radiation delivery unit 17 may also irradiate radiation R to the lesion of a user P sitting in a chair. In this embodiment, the user P is a user with a lesion such as a tumor in the chest or abdomen. In this embodiment, the radiation delivery unit 17 irradiates radiation R to the lesion of the user P's chest or abdomen. In this embodiment, the radiation R is X-rays. The radiation delivery unit 17 includes an accelerator tube (not shown) that accelerates electrons generated by an electron gun (not shown) and a metal target (not shown) on which the electrons accelerated by the acceleration tube collide. When the accelerated electrons collide with the metal target, X-rays are emitted (radiation R). A multi-leaf collimator (not shown) is attached to the exit side of the radiation delivery unit 17. The multi-leaf collimator supports multiple leaves made of an X-ray shielding material so that they can be individually moved. By moving the multiple metal leaves, an irradiation field of any shape can be formed. The radiation R may be a proton beam or a heavy particle beam.

The radiation delivery unit 17 has a drive mechanism (not shown). The drive mechanism can freely adjust the irradiation direction of the radiation R irradiated from the radiation delivery unit 17. This allows the radiation delivery unit 17 to adjust the irradiation direction of the radiation R in accordance with the movement of the lesion. The radiation delivery unit 17 can also adjust the delivery timing of the radiation R and the stop timing of the radiation R. In other words, the radiation delivery unit 17 can adjust the delivery period of the radiation R. Note that the radiation R delivered by the radiation delivery unit 17 is not limited to X-rays. The radiation R may be other radiation such as an electron beam or a particle beam.

The radiation control unit 13 is capable of communicating with each of the measurement block 19, the radiation delivery unit 17, and the control block 34 of the respiratory stabilization device 30. The radiation control unit 13 does not have to be capable of communicating with the control block 34. The radiation control unit 13 may be capable of communicating with each of the measurement block 19, the radiation delivery unit 17, and the control block 34 via wired communication means or wireless communication means. The radiation control unit 13 controls a drive mechanism (not shown) of the radiation delivery unit 17 based on at least one of respiratory cycle information Ib transmitted from the respiratory stabilization device 30, the respiratory cycle Tb transmitted from the measurement block 19, and the respiratory cycle Tb transmitted from the control block 34. This allows the radiation delivery unit 17 to adjust the delivery direction of radiation R based on at least one of the respiratory cycle information Ib and the respiratory cycle Tb. The radiation control unit 13 is also capable of adjusting the timing at which the radiation delivery unit 17 irradiates radiation R and the timing at which it stops irradiating radiation R based on at least one of the respiratory cycle information Ib and the respiratory cycle Tb. This allows the radiation delivery unit 17 to adjust the delivery period of radiation R based on at least one of the respiratory cycle information Ib and the respiratory cycle Tb.

In this embodiment, the radiation control unit 13 and the control block 34 are computers that control the operation of each unit of the radiation delivery system 10. A control program that controls the operation of each unit is installed in the radiation control unit 13 and the control block 34. At least a part of the functions of each component of the radiation control unit 13 and the control block 34 is realized by, for example, a processor such as a CPU (Central Processing Unit) executing a control program, i.e., software, stored in a memory unit (not shown).

At least a part of the functions of the components of the radiation control unit 13 and the control block 34 can be implemented using, for example, a large scale integration (LSI), an application specific integrated circuit (ASIC), a field programmable gate array (FPGA), and a graphics processing unit (GPU), or may be realized by a combination of software and hardware. The radiation control unit 13 and the control block 34 may include a memory unit (not shown). In this case, the memory unit (not shown) may be a RAM, a ROM, a hard disk drive (HDD), and a flash memory.

The respiratory stabilization device 30 is a medical device that stabilizes the respiratory cycle at an optimal cycle for each user P. As shown in FIG. 1, the respiratory stabilization device 30 includes an input block 31, a control block 34, an electrical stimulator 38, a display unit 41, a sound unit 42, and a stimulation interrupt unit 45.

The input block 31 inputs respiratory cycle information Ib, which is previously acquired information on the respiratory cycle of the user P, to the control block 34. The input block 31 is capable of communicating with the control block 34. In this embodiment, the input block 31 includes an input unit 31a, breath sound sensor 31c, flow rate sensor 31d, displacement sensor 31e, and a micro controller unit 31g. Note that the input block 31 does not necessarily have to include at least one of the breath sound sensor 31c, flow rate sensor 31d, and displacement sensor 31e.

In this embodiment, “previously acquired” means “obtained before the lesion is treated by irradiating the user P with radiation R using the radiation delivery device 11.” Therefore, in this embodiment, “respiratory cycle information Ib” is “information on the respiratory cycle of user P obtained before the lesion is treated by irradiating the user P with radiation R using the radiation delivery device 11.” The respiratory cycle information Ib includes each of an exhalation period Td during which the user P exhales air, and an inhalation period Ti during which the user P inhales air.

The input device 31a shown in FIGS. 1 and 2 receives respiratory cycle information Ib of the user P, which was acquired in advance, and inputs the respiratory cycle information Ib to the control unit 34. As shown in FIG. 1, the input device 31a also inputs an aimed current value In of the current Ic, which has been input in advance, to the control block 34. In this embodiment, the input unit 31a may be, for example, a personal computer, a tablet terminal, or a smartphone. As shown in FIG. 2, the input unit 31a is a personal computer. A medical professional W may input the respiratory cycle information Ib and the aimed current value In to the input unit 31a, or the user P may input the respiratory cycle information Ib and the aimed current value In to the input device 31a. In this embodiment, the input device 31a is placed in an operation room Rc adjacent to the treatment room Rt. The input unit 31a may also be placed in the treatment room Rt.

The breath sound sensor 31c shown in FIG. 1 detects the breath sounds of the user P. More specifically, the breath sound sensor 31c detects breath sounds when the user P inhaled air and when the user P exhaled air. In this manner, the breath sound sensor 31c provides the breathing cycle information of the user P. In this embodiment, the breath sound sensor 31c is, for example, a microphone. The breath sound sensor 31c communicates with the micro controller unit 31g, where the breath sound sensor 31c outputs electrical signals and transmits the electrical signals to the micro controller unit 31g.

The flow rate sensor 31d detects the flow rate of air due to breathing of the user P. More specifically, the flow rate sensor 31d detects the flow rate of air near the nose and mouth of the user P when the user P inhales air, and the flow rate of air near the nose and mouth of the user P when the user P exhales air. In this manner, the flow rate sensor 31d gives the breathing cycle information of the user P. In this embodiment, the flow rate sensor 31d is, for example, an anemometer. The flow rate sensor 31d communicates with the micro controller unit 31g, where the flow rate sensor 31d outputs an electrical signal and transmits it to the micro controller unit 31g.

The displacement sensor 31e detects the displacement of the abdomen of the user P. More specifically, the displacement sensor 31e detects the displacement of the abdomen due to abdominal expansion when the user P inhales air and abdominal contraction when the user P exhales air. In this way, the displacement sensor 31e gives the respiratory cycle information of the user. In this embodiment, the displacement sensor 31e is, for example, a laser displacement sensor that irradiates the abdomen of the user P with infrared rays and measures the position and displacement of the abdomen from the reflected light. The displacement sensor 31e communicates with the micro controller unit 31g, where the displacement sensor 31e outputs an electrical signal and transmits it to the micro controller unit 31g. Note that the configuration of the displacement sensor 31e is not limited to that of this embodiment, and may be based on a different measuring device such as an acceleration sensor.

The micro controller unit 31g is a microprocessor such as an MCU (Microcontroller Unit), that can read each of the breath sound sensor 31c, the flow rate sensor 31d, the displacement sensor 31e, and can also communicate with the control block 34 via wired communication means such as a cable, or wireless communication means such as a wireless LAN.

The micro controller unit 31g derives respiratory cycle information Ib of the user P based on the electrical signals transmitted from the breath sound sensor 31c, the flow rate sensor 31d, and the displacement sensor 31e. As a result, the input block 31 derives respiratory cycle information Ib based on the breath sounds of the user P, the air flow rate due to the user P's breathing, and the movement of the user P's abdomen. The micro controller unit 31g inputs the derived respiratory cycle information Ib of the user P to the control block 34. Note that the micro controller unit 31g may derive the respiratory cycle information Ib of the user P based on the electrical signals transmitted from any one of the breath sound sensor 31c, the flow rate sensor 31d, and the displacement sensor 31e. The input block 31 does not necessarily have to include the micro controller unit 31g. In this case, the breath sound sensor 31c, the flow rate sensor 31d, and the displacement sensor 31e transmit electrical signals to the input device 31a, and the input unit 31a derives the respiratory cycle information Ib of the user P.

As shown in FIG. 2, the electrical stimulator 38 is attached to the abdomen of the user P and provides electrical stimulation to the abdomen. In this embodiment, the electrical stimulator 38 is a conductive electrode. The electrical stimulator 38 can be made of a conductive material such as metal, conductive fiber, or conductive rubber. The electrical stimulator 38 is electrically connected to the control unit 34 via a cable 39. As shown in FIG. 1, a current Ic with a predetermined waveform is supplied from the control block 34 to the electrical stimulator 38. As a result, when a current flows through the abdomen of the user P via the electrical stimulator 38, electrical stimulation is provided to the abdomen of the user P.

As shown in FIG. 2, the respiratory stabilization device 30 has multiple electrical stimulators 38. In this embodiment, the respiratory stabilization device 30 has two electrical stimulators 38. The number of electrical stimulators 38 included in the respiratory stabilizing device 30 may be one or three or more. As shown in FIG. 3, in this embodiment, each electrical stimulator 38 is attached to the rectus abdominis muscle of the user P. This makes it easier to force the user P to breathe through electrical stimulation. Note that the attachment locations of each electrical stimulator 38 are not limited to those described in this embodiment. For example, one electrical stimulator 38 may be attached to a muscle other than the rectus abdominis muscle, such as the external oblique muscle and the internal oblique muscle, or both electrical stimulators 38 may be attached to muscles other than the rectus abdominis muscle, such as the external oblique muscle and the internal oblique muscle.

The control block 34 shown in FIG. 1 controls the current Ic supplied to the electrical stimulator 38 based on respiratory cycle information Ib that is input from the input block 31. More specifically, the control block 34 controls the waveform of the current Ic supplied to the electrical stimulator 38. The control block 34 is electrically connected to a power source (not shown). This supplies power to the control block 34. In this embodiment, the power source may be a battery, a rechargeable battery, or a commercial power source. The control block 34 includes a control unit 35 and a current supply unit 36. In this embodiment, the control block 34 is also capable of controlling the current Ic based on the respiratory cycle Tb that is input from the measurement block 19.

The control unit 35 is capable of communicating with each of the input block 31, the measurement block 19, the current supply unit 36, and the radiation control unit 13. This allows each of the input block 31 and the measurement block 19 to communicate with the control block 34. The control unit 35 may be capable of communicating with each of the input block 31, the measurement block 19, the current supply unit 36, and the radiation control unit 13 via wired communication means or wireless communication means. The control unit 35 receives the respiratory cycle information Ib and the target current value In that are coming from the input block 31. The control unit 35 also receives the respiratory cycle Tb from the measurement block 19. Note that the control unit 35 does not necessarily have to be capable of communicating with the radiation control unit 13.

Based on the respiratory cycle information Ib, the control unit 35 derives a first period T1 during which current Ic is supplied to the electrical stimulator 38, and a second period T2 during which the supply of current Ic to the electrical stimulator 38 is stopped. In this embodiment, the length of the first period T1 is the same as the length of the exhalation period Td included in the respiratory cycle information Ib, and the length of the second period T2 is the same as the length of the inhalation period Ti included in the respiratory cycle information Ib. Also, in this embodiment, the control unit 35 may derive the first period T1 and the second period T2 based on the respiratory cycle Tb. In this case, the length of the first period T1 is the same as the length of the period during which the user P exhales air, and the length of the second period T2 is the same as the length of the period during which the user P inhales air. The control unit 35 transmits the first period T1, the second period T2, and the aimed current value In to the current supply unit 36.

The current supply unit 36 generates a current Ic and supplies the current Ic to the electrical stimulator 38 via a cable 39. In the present embodiment, the current supply unit 36 is an AC current generator. The current supply unit 36 is capable of adjusting the first period T1, the second period T2, and a supply current value Is, which is a current value supplied to the electrical stimulator 38 based on the first period T1, the second period T2, and the aimed current value In, each being transmitted from the control unit 35. This allows the control unit 34 to adjust the first period T1 and the second period T2 based on the respiratory cycle information Ib. The control block 34 is also capable of adjusting the first period T1 and the second period T2 based on the respiratory cycle Tb.

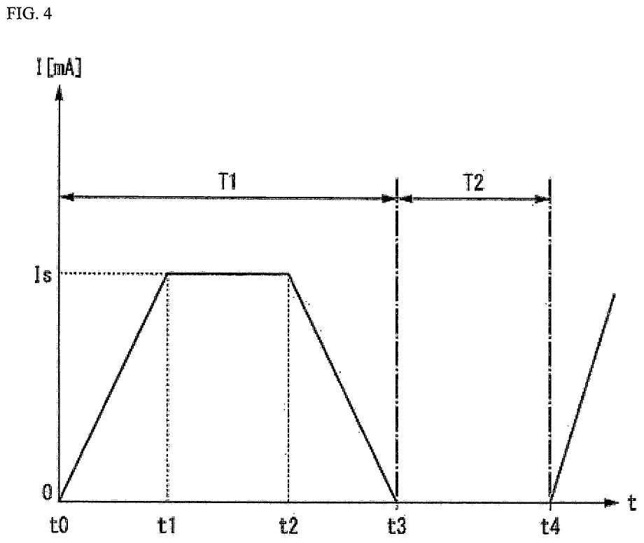

FIG. 4 is a diagram showing an example of the waveform of current Ic in this embodiment. The horizontal axis of FIG. 4 represents time t. The vertical axis of FIG. 4 represents current value I. The current supply unit 36 generates current during a first period T1 and does not generate current during a second period T2. As a result, current is supplied to the electrical stimulator 38 during the first period T1, and the supply of current to the electrical stimulator 38 is stopped during the second period T2. As a result, electrical stimulation is applied to the abdomen of the user P during the first period T1, causing the user P to exhale air. Furthermore, as no electrical stimulation is applied to the abdomen of the user P during the second period T2, the user P inhales air. Although not shown, the first period T1 and the second period T2 are alternately repeated. As a result, the user P can breathe at a constant cycle, thereby stabilizing the breathing cycle. As described above, the length of the first period T1 is the same as the length of the exhalation period Td included in the respiratory cycle information Ib, and the length of the second period T2 is the same as the length of the inhalation period Ti included in the respiratory cycle information Ib. Therefore, in this embodiment, the respiratory cycle can be stabilized at an optimal period for each user P. In FIG. 4, the first period T1 is the period from time t0 to t3, and the second period T2 is the period from time t3 to t4. The length of the first period T1 may be the same as the period included in the respiratory cycle Tb during which the user P exhales air, and the length of the second period T2 may be the same as the period included in the respiratory cycle Tb during which the user P inhales air.

In this embodiment, the current waveform during the first period T1 is approximately trapezoidal. More specifically, the current waveform rises from 0 mA to a supply current value Is between times t0 and t1, remains constant at the supply current value Is between times t1 and t2, and then drops to 0 mA between times t2 and t3. As a result, the current value gradually increases between times to and t1, and the electrical stimulation applied to the abdomen of the user P gradually increases. Furthermore, the current value gradually decreases between times t2 and t3, and the electrical stimulation applied to the abdomen of the user P gradually decreases. This prevents the user P from experiencing increased pain due to the electrical stimulation.

The current waveform in the first period T1 may be rectangular. In this case, the current waveform rises to the supply current value Is at time to, remains constant at the supply current value Is between time to and t3, and then drops to 0 mA at time t3. The current waveform in the first period T1 may also be other shapes, such as a semicircular arc. The current waveform in the first period T1 can be determined appropriately considering the pain of the user P and the stability of the breathing cycle, etc.

As shown in FIG. 1, the control unit 35 transmits the respiratory cycle information Ib of the user P and the respiratory cycle Tb of the user P to the radiation control unit 13. As described previously, the measurement control unit 19e transmits the respiratory cycle Tb of the user P to the radiation control unit 13. Therefore, the radiation control unit 13 can control a drive mechanism (not shown) of the radiation delivery unit 17 based on at least one of the respiratory cycle information Ib transmitted from the respiratory stabilization device 30 and the respiratory cycle Tb measured by the measurement unit 19. This allows the radiation delivery unit 17 to adjust the direction of irradiation of the radiation R based on at least one of the respiratory cycle information Ib and the respiratory cycle Tb. Note that the measurement control unit 19e may not need to transmit the respiratory cycle Tb of the user P to the radiation control unit 13.

If the lesion is located in the chest or abdomen of the user P, the position of the lesion will move as the user P breathes. In this embodiment, as described above, the respiratory stabilization device 30 can stabilize the respiratory cycle of the user P, thereby stabilizing the movement cycle of the lesion area. This allows the radiation delivery unit 17 to adjust the direction in which the radiation R is irradiated in synchronization with the respiratory cycle of the user P. This makes it possible to prevent the radiation R from being irradiated to areas of the user P other than the lesion area. Furthermore, the time for treating the lesion can be shortened.

Furthermore, as described above, the radiation delivery unit 17 can adjust the time for delivering the radiation R. Therefore, the radiation delivery unit 17 can, for example, deliver the radiation R when the user P inhales air and stop delivering the radiation R when the user P exhales air. This allows the radiation delivery unit 17 to deliver the radiation R to a predetermined position where the lesion is located during the user P's inhalation period. In other words, the radiation delivery unit 17 can deliver radiation R to the predetermined position when the lesion is located there. In this case, the delivery of radiation R to areas other than the lesion of the user P can also be prevented. Furthermore, the treatment time for the lesion can be shortened. Note that the radiation delivery unit 17 may conversely deliver the radiation R when the user P exhales air and stop delivering the radiation R when the user P inhales air. Even in this case, the radiation delivery unit 17 can deliver the radiation R to a predetermined position when the lesion is located there. That is, the radiation delivery unit 17 can irradiate the predetermined position with the radiation R in synchronization with the timing at which the lesion is positioned at the predetermined position. Therefore, it is possible to prevent the radiation R from being irradiated to areas of the user P other than the lesion area.

The stimulation interrupt unit 45 is capable of communicating with the control block 34. The stimulation interrupt unit 45 may be capable of communicating with the control unit 35 via wired or wireless communication means. As shown in FIG. 2, the user P holds the stimulation interrupt unit 45 while the respiratory stabilization device 30 is operating. The stimulation interrupt unit 45 has a button (not shown). When the user P presses the button, the stimulation interrupt unit 45 transmits a stop signal St to the control block 34, as shown in FIG. 1. That is, the stimulation interrupt unit 45 transmits the stop signal St to the control block 34 through the user P's operation. Upon receiving the stop signal St, the control block 34 stops the supply of the current Ic to the electrical stimulator 38 through the current supply unit 36. This allows the user P to quickly stop the supply of the current Ic to the electrical stimulator 38 when the user P feels pain or discomfort in response to the electrical stimulation or the forced breathing at the respiratory cycle given by the electrical stimulation. Therefore, the safety of the user P can be suitably ensured.

In this embodiment, in addition to the user P, a medical professional W operating the radiation delivery device 11 in the operation room Rc may hold the stimulation interrupt unit 45 during operation of the respiratory stabilization device 30. In this way, if the medical professional W notices something unusual about the user P, they can operate the stimulation interrupt unit 45 to quickly stop the supply of the current Ic from the current supply unit 36 to the electrical stimulator 38. This makes it possible to more appropriately ensure the safety of the user P.

The display unit 41 displays an instruction image Vs that is visible to the user P. As shown in FIG. 1, the display unit 41 is capable of communicating with the control block 34. The display unit 41 may be capable of communicating with the control unit 35 via wired communication means or wireless communication means. In this embodiment, the display unit 41 is a display such as goggles with a display that can be worn on the head of the user P, or the like.

The instruction video Vs is an image synchronized with the first period T1 and the second period T2. The instruction video Vs includes a first instruction video Vs1 and a second instruction video Vs2. The first instruction video Vs1 is displayed during the first period T1. The first instruction video Vs1 is an image instructing the user P to exhale air. The second instruction video Vs2 is displayed during the second period T2. The second instruction video Vs2 is an image instructing the user P to inhale air. In this embodiment, the first instruction video Vs1 is a video of a person or an animated character exhaling air, and the second instruction video Vs2 is a video of a person or an animated character inhaling air. This makes it possible to more effectively stabilize the breathing cycle of the user P. Note that the instruction video Vs is not limited to this embodiment and may be any image that can stabilize the breathing cycle of the user P.

The sound unit 42 shown in FIG. 2 generates an instruction sound Ss that can be heard by the user P. As shown in FIG. 1, the sound unit 42 is capable of communicating with the control block 34. The sound unit 42 may be capable of communicating with the control unit 35 via wired communication means or wireless communication means. In this embodiment, the sound unit 42 is a speaker. The sound unit 42 may also be earphones that can be worn on the ears of the user P, headphones that can be worn on the head of the user P, or the like.

The instruction sounds Ss are sounds synchronized with the first period T1 and the second period T2. The instruction sounds Ss include a first instruction sound Ss1 and a second instruction sound Ss2. The first instruction sound Ss1 is generated during the first period T1. The first instruction sound Ss1 is a sound instructing the user P to exhale air. The second instruction sound Ss2 is generated during the second period T2. The second instruction sound Ss2 is a sound instructing the user P to inhale air. In this embodiment, the first instruction sound Ss1 is a human voice saying “Please exhale,” and the second instruction sound Ss2 is a human voice saying “Please inhale.” This makes it possible to more effectively stabilize the breathing cycle of the user P. Note that the instruction sounds Ss are not limited to those in this embodiment and may be any sound that can stabilize the breathing cycle of the user P.

The respiratory stabilization device 30 does not need to include both the display unit 41 and the sound unit 42, and may include only one of the display unit 41 or the sound unit 42.

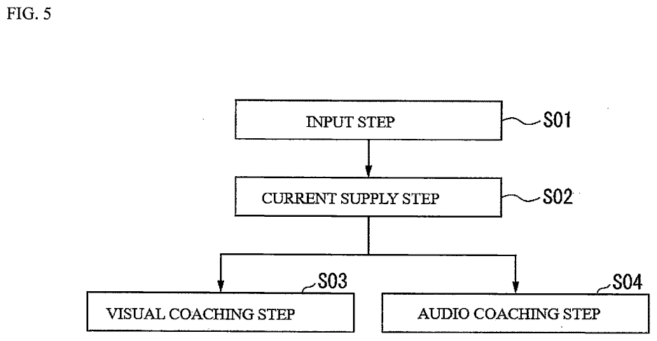

FIG. 5 is a flowchart showing a respiratory stabilization method of this embodiment, where the respiratory stabilization method is described. The respiratory stabilization method of this embodiment is a method for stabilizing the respiratory cycle of a user P using a respiratory stabilization device 30. As shown in FIG. 5, the respiratory stabilization method of this embodiment includes an input step S01 in which respiratory cycle information Ib, which is the previously acquired respiratory cycle of the user P, is input to the control block 34 from the input block 31, a current supply step S02 in which the control block 34 supplies a current Ic to the electrical stimulator 38, a visual coaching step S03 in which the display unit 41 causes the user P to view an instruction image Vs, and an audio coaching step S04 in which the sound unit 42 causes the user P to hear an instruction sound Ss.

In the input step S01, respiratory cycle information Ib, which is the respiratory cycle of the user P acquired in advance, is input to the control block 34 via the input block 31. In the input step S01 of this embodiment, a medical professional W inputs the respiratory cycle information Ib of the user P acquired in advance to the input device 31a, and inputs the respiratory cycle information Ib to the control unit 34 via the input unit 31a. The medical professional W can use the breath sound sensor 31c, the flow rate senor 31d, and the displacement sensor 31e to acquire the respiratory cycle information Ib of the user P, for example. Note that the person who inputs the respiratory cycle information Ib to the input device 31a is not limited to the medical professional W and may be, for example, the user P. Furthermore, the respiratory cycle information Ib may be acquired by any of the breath sound sensor 31c, the flow rate sensor 31d, and the displacement sensor 31e, and then, the respiratory cycle information Ib can be sent to the control block 34 via the micro controller unit 31g. When the respiratory cycle information Ib is sent to the control block 34, the input step S01 ends.

In the input step S01, the measurement control unit 19e may input the breathing cycle Tb of the user P to the control unit 34. In this case, the control unit 35 derives the first period T1 and the second period T2 based on the breathing cycle Tb of the user P. In this case, the length of the first period T1 is the same as the period included in the breathing cycle Tb during which the user P exhales air, and the length of the second period T2 is the same as the period included in the breathing cycle Tb during which the user P inhales air.

In the current supply step S02, the control block 34 supplies the current Ic to the electrical stimulator 38. In the current supply step S02, as shown in FIG. 2, the user P first lies on the couch 18. The user P may be sitting in a chair or standing. Next, the medical professional W attaches the electrical stimulator 38 to the abdomen of the user P. Next, the medical professional W starts the operation of the respiratory stabilization device 30. As a result, the current Ic is supplied to the electrical stimulator 38 during the first period T1, and the supply of the current Ic to the electrical stimulator 38 is stopped during the second period T2. Therefore, as described above, the electrical stimulation makes it easier for the user P to exhale air during the first period T1 and to inhale air during the second period T2. This allows the user P's respiratory cycle to be stabilized. When the current Ic is supplied to the electrical stimulator 38, the current supply step S02 ends.

In the visual coaching step S03, the user P is made to view the instruction video Vs on the display unit 41. In the visual coaching step S03, the display unit 41 displays the instruction video Vs, which is an image synchronized with the first period T1 and the second period T2. This makes it possible to more effectively stabilize the breathing cycle of the user P, as described earlier.

In the audio coaching step S04, the user P hears the indicator sound Ss by the sound unit 42. In the audio coaching step S04, the sound unit 42 generates the indicator sound Ss, which is a sound synchronized with the first period T1 and the second period T2. As a result, the breathing cycle of the user P is more effectively stabilized as described above,

The respiratory stabilization method does not have to include both of the visual coaching step S03 and the audio coaching step S04. Even in this case, the available unit can more effectively stabilize the respiratory cycle of the user P. Furthermore, the respiratory stabilization method can exclude both the visual coaching step S03 and the audio coaching step S04. Even in this case, the respiratory cycle of the user P can be stabilized by the periodic electrical stimulation applied to the abdomen of the user P.

Once the respiratory cycle of the user P has been stabilized by the above-described respiratory stabilization method, the medical professional W starts irradiating the lesion with radiation R using the radiation delivery device 11. At this time, as described above, the respiratory cycle of the user P is stable, and therefore the movement cycle of the lesion is also stable. This allows the radiation delivery unit 17 to adjust the direction of irradiation of radiation R in synchronization with the respiratory cycle of the user P, as described above. Furthermore, as described above, the radiation delivery unit 17 can irradiate radiation R to a predetermined position in synchronization with the timing when the lesion is positioned at a predetermined position. This makes it possible to prevent radiation R from being irradiated to areas of the user P other than the lesion area.

According to this embodiment, the respiratory stabilization device 30 includes an electrical stimulator 38 attached to the abdomen of the user P and electrically stimulating the abdomen, the control block 34 controlling the current Ic supplied to the electrical stimulator 38, and an input block 31 capable of communicating with the control block 34 and inputting respiratory cycle information Ib representing the previously acquired respiratory cycle of the user P to the control block 34. The control block 34 is capable of adjusting a first period T1 during which the current Ic is supplied to the electrical stimulator 38 and a second period T2 during which the supply of the current Ic to the electrical stimulator 38 is stopped, based on the respiratory cycle information Ib. Therefore, because the first period T1 and the second period T2 can be adjusted based on the previously acquired respiratory cycle information Ib of the user P, the respiratory cycle can be stabilized at an optimal period for each user P. This reduces the burden on the user P.

When stabilizing the breathing cycle of a user P using a ventilator, the breathing cycle of the user P is forcibly stabilized by forcibly introducing air through a tube held by the user P. In this case, the user P is likely to feel fear or pain when breathing at the breathing cycle set by the ventilator, making it difficult for the user P to breathe in a relaxed state, and making it difficult to ensure the safety of the user P. In contrast, in this embodiment, the breathing cycle of the user P is stabilized by electrical stimulation, so if the user P feels pain or the like about breathing at the breathing cycle set by the electrical stimulation, they are likely to breathe spontaneously, deviating from the breathing cycle set by the electrical stimulation. Therefore, in this embodiment, it is easy to ensure the safety of the user P.

According to this embodiment, the respiratory cycle information Ib includes an exhalation period Td during which the user P exhales air and an inhalation period Ti during which the user P inhales air, with the length of the first period T1 being the same as the exhalation period Td, and the length of the second period T2 being the same as the inhalation period Ti. Therefore, the length of the first period T1 during which current Ic is supplied to the electrical stimulator 38 and the length of the second period T2 during which the supply of current Ic to the electrical stimulator 38 is stopped can be accurately matched with the length of the exhalation period Td during which the user P exhales air and the length of the inhalation period Ti during which the user P inhales air, respectively. This more effectively stabilizes the respiratory cycle at an optimal period for each user P. This more effectively reduces the burden on the user.

According to this embodiment, the input block 31 has a breath sound sensor 31c that acquires the breath sounds of the user P and derives respiratory cycle information Ib based on the breath sounds. Therefore, compared to when respiratory cycle information Ib is acquired by attaching a measurement device to the user P, respiratory cycle information Ib of a user P in a relaxed state can be acquired. This allows the input block 31 to accurately acquire the length of the exhalation period Td, which is the period during which the user P exhales air, and the length of the inhalation period Ti, which is the period during which the user P inhales air. Therefore, it is possible to more preferably stabilize the respiratory cycle at an optimal period for each user P.

According to this embodiment, the input block 31 has a flow rate sensor 31d that acquires the air flow rate due to breathing by the user P, and derives respiratory cycle information Ib based on the air flow rate. Therefore, compared to acquiring respiratory cycle information Ib by attaching a measurement device to the user P, it is possible to acquire respiratory cycle information Ib of a user P in a relaxed state. This allows the input block 31 to accurately acquire the lengths of the exhalation period Td and the inhalation period Ti. Therefore, it is possible to more preferably stabilize the respiratory cycle at an optimal period for each user P.

According to this embodiment, the input block 31 has a displacement sensor 31e that acquires the displacement of the abdomen of the user P and derives respiratory cycle information based on the abdominal displacement. The period of the displacement of the abdomen of the user P is precisely linked to the respiratory cycle of the user P, so the input block 31 can accurately acquire the length of each of the exhalation period Td and the inhalation period Ti. Therefore, it is possible to more preferably stabilize the respiratory cycle at an optimal period for each user P.

According to this embodiment, the respiratory stabilization device 30 includes a stimulation interrupt unit 45 that transmits a stop signal St to the control block 34 in response to an operation by the user P. Upon receiving the stop signal St, the control block 34 stops the supply of the current Ic to the electrical stimulator 38. Therefore, if the user P feels pain or the like in response to the electrical stimulation or if the user P feels pain or the like in response to breathing at the respiratory cycle given by the electrical stimulation, the supply of the current Ic to the electrical stimulator 38 can be quickly stopped. This makes it possible to more appropriately ensure the safety of the user P.

According to this embodiment, the respiratory stabilization device 30 includes a display unit 41 that displays an instruction video Vs that is visible to the user P. The instruction video Vs includes a first instruction video Vs1 that is displayed during a first period T1 and instructs the user P to exhale air, and a second instruction video Vs2 that is displayed during a second period T2 and instructs the user P to inhale air. Thus, by viewing the instruction video Vs during operation of the respiratory stabilization device 30, the user P is more likely to exhale air during the first period T1 and to inhale air during the second period T2. This makes it possible to more effectively stabilize the respiratory cycle at an optimal cycle for each user P.

According to this embodiment, the respiratory stabilization device 30 includes a sound unit 42 that generates instruction sounds Ss that are audible to the user P. The instruction sounds Ss include a first instruction sound Ss1 that is generated in a first period T1 and instructs the user P to exhale air, and a second instruction sound Ss2 that is generated in a second period T2 and instructs the user P to inhale air. Thus, by hearing the instruction sounds Ss during operation of the respiratory stabilization device 30, the user P is more likely to exhale air in the first period T1 and to inhale air in the second period T2. This makes it possible to more preferably stabilize the respiratory cycle at an optimal cycle for each user P.

According to the present embodiment, the electrical stimulator 38 is attached to at least the rectus abdominis muscle of user P. Therefore, electrical stimulation is applied to the rectus abdominis muscle of user P, making it easier to force breathing of user P. Therefore, the respiratory cycle of each user P can be more suitably stabilized.

According to this embodiment, the control block 34 can adjust the supply current value Is, i.e., the value of the current supplied to the electrical stimulator 38.

Therefore, the intensity of the electrical stimulation can be adjusted for each user P. As a result, if the user P is, for example, an infant or other person with low tolerance for electrical stimulation, the electrical stimulation applied to the user P can be weakened. Therefore, the safety of the user P can be more appropriately ensured. Furthermore, if the user P's respiratory cycle deviates from the cycle determined by the first period T1 and the second period T2, the electrical stimulation applied to the user P can be strengthened. This more appropriately stabilizes the respiratory cycle at an optimal cycle for each user P.

According to this embodiment, the radiation delivery system 10 includes a respiratory stabilization device 30 and a radiation delivery unit 17 that irradiates radiation R to a lesion in the chest or abdomen of the user P. The radiation delivery unit 17 is capable of adjusting at least one of the delivery direction of radiation R or the delivery time of radiation R based on respiratory cycle information Ib. As described above, the respiratory stabilization device 30 can stabilize the respiratory cycle of the user P. Therefore, as described above, the movement cycle of the lesion in the chest or abdomen of the user P can be stabilized. This allows the radiation delivery unit 17 to adjust the delivery direction of radiation R in synchronization with the respiratory cycle of the user P. Furthermore, as described above, the radiation delivery unit 17 can deliver radiation R to a predetermined position in synchronization with the timing when the lesion is positioned there. Therefore, irradiation of radiation R to areas other than the lesion of the user P can be prevented. This can improve treatment accuracy and shorten the treatment time for the lesion area.

According to this embodiment, the radiation delivery system 10 includes a respiratory stabilization device 30, a radiation delivery unit 17 that delivers radiation R to a lesion in the chest or abdomen of the user P, and a measurement block 19 that measures the user P's respiratory cycle Tb. The control block 34 can adjust the first period T1 and the second period T2 based on the respiratory cycle Tb, and the radiation delivery unit 17 can adjust at least one of the delivery direction of radiation R or the delivery time of radiation R based on the respiratory cycle Tb measured by the measurement block 19. Therefore, since the first period T1 and the second period T2 can be adjusted based on the user P's respiratory cycle when treating the lesion area, the respiratory cycle can be stabilized at an optimal cycle for each user P. This reduces the burden on the user P. The radiation delivery unit 17 can adjust the delivery direction of radiation R in synchronization with the respiratory cycle Tb. Furthermore, the radiation delivery unit 17 can irradiate radiation R to a predetermined position in synchronization with the timing when the lesion is positioned there. Therefore, as described above, the delivery of radiation R to areas other than the lesion of the user P can be prevented. In other words, the accuracy of treatment can be improved and the time required to treat the lesion can be reduced.

According to this embodiment, the respiratory stabilization method uses a respiratory stabilization device 30 including an electrical stimulator 38 attached to the abdomen of a user P and electrically stimulating the abdomen, a control block 34 controlling the current Ic supplied to the electrical stimulator 38, and an input block 31 capable of communicating with the control block 34. The method includes an input step S01 in which the input block 31 inputs previously acquired respiratory cycle information Ib representing the respiratory cycle of the user P to the control block 34, and a current supply step S02 in which the control unit 34 supplies the current Ic to the electrical stimulator 38. In the current supply step S02, the control block 34 can adjust a first period T1 during which the current Ic is supplied to the electrical stimulator 38 and a second period T2 during which the supply of the current Ic to the electrical stimulator 38 is stopped, based on the respiratory cycle information Ib. Therefore, as described above, the first period T1 and the second period T2 can be adjusted based on the previously acquired respiratory cycle information Ib of the user P, thereby stabilizing the respiratory cycle at an optimal period for each user P. This reduces the burden on the user.

Although the embodiments of the present invention were described above, the configurations and combinations in the embodiments are merely examples, and additions, omissions, substitutions, and other modifications of the configurations are possible without departing from the spirit of the present invention. Furthermore, the present invention is not limited to the embodiments disclosed here.

The present technology can be configured as follows.
(1) A respiratory stabilization device comprising: an electrical stimulator attached to the abdomen of a user and applying electrical stimulation to the abdomen; a control block that controls the current supplied to the electrical stimulator; and an input block that is capable of communicating with the control block and inputs the user's respiratory cycle information acquired in advance to the control unit, wherein the control block is capable of adjusting a first period during which the current is supplied to the electrical stimulator and a second period during which the supply of the current to the electrical stimulator is stopped based on said respiratory cycle information.
(2) The respiratory stabilization device of (1), wherein said respiratory cycle information includes an exhalation period during which the user exhales air and an inhalation period during which the user inhales air, and the length of said first period is the same as the length of the exhalation period, and the length of said second period is the same as the length of the inhalation period.
(3) The respiratory stabilization device of (1) or (2), wherein the input block has a breath sound sensor that acquires breath sounds of the user and derives said respiratory cycle information based on the breath sounds.
(4) The respiratory stabilization device of (1) or (2), wherein the input block has a flow rate sensor that acquires an air flow rate due to breathing of the user and derives said respiratory cycle information based on the air flow rate.
(5) The respiratory stabilization device of (1) or (2), wherein the input block has a displacement sensor that acquires a displacement of the user's abdomen and derives said respiratory cycle information based on the displacement of the abdomen.
(6) A respiratory stabilization device in any one of (1) to (5), further comprising a stimulation interrupt unit that transmits a stop signal to the control block by the operation of the user, wherein the control block stops supplying the current to the electrical stimulator when it receives the stop signal.
(7) A respiratory stabilization device in any one of (1) to (6), further comprising a display unit that displays instruction images visible to the user, wherein the instruction images including a first instruction image that is displayed during the first period and instructs the user to exhale air, and a second instruction image that is displayed during the second period and instructs the user to inhale air.
(8) A respiratory stabilization device in any one of (1) to (7), further comprising a sound unit that generates instruction sounds audible to the user, wherein the instruction sounds including a first instruction sound that is generated during the first period and instructs the user to exhale air, and a second instruction sound that is generated during the second period and instructs the user to inhale air;
(9) The respiratory stabilization device in any one of (1) to (8), wherein the electrical stimulator is attached to at least the rectus abdominis muscle of the user.
(10) A respiratory stabilization device in any one of (1) to (9), wherein the control block is capable of adjusting the current intensity supplied to said electrical stimulator.
(11) A radiation delivery system comprising: said respiratory stabilization device according to any one of (1) to (10), and a radiation delivery unit that delivers radiation to a lesion of the chest or a lesion of the abdomen of the user, wherein said radiation delivery unit is capable of adjusting at least one of the delivery direction the radiation or the delivery time of the radiation based on said respiratory cycle information.
(12) A radiation delivery system comprising: said respiratory stabilization device according to any one of (1) to (10); a radiation delivery unit that delivers radiation to a lesion in the chest or a lesion in the abdomen of the user; and a measurement block that measures the respiratory cycle of the user, wherein said control block in said respiratory stabilization device is capable of adjusting the first period and the second period based on the respiratory cycle; and said radiation delivery unit is capable of adjusting at least one of the delivery direction or the delivery time during which the radiation is delivered based on the respiratory cycle measured by the measurement block.
(13) A respiratory stabilization method using a respiratory stabilization device comprising an electrical stimulator attached to a user's abdomen and providing electrical stimulation to the abdomen, a control block controlling the current supplied to the electrical stimulator, and an input block capable of communicating with the control block, wherein the respiratory stabilization method comprising: an input step in which said respiratory cycle information representing the user's respiratory cycle, which has been previously acquired, is sent to the control block from the input block, a current supply step in which the control block supplies the current to the electrical stimulator, wherein the control block is capable of adjusting a first period during which the current is supplied to said electrical stimulator and a second period during which the current supply to the electrical stimulator is stopped based on said respiratory cycle information.

What is claimed is:

1. A respiratory stabilization device, comprising:
an electrical stimulator attached to an abdomen of a user and applying electrical stimulation to said abdomen;
a control block that controls a current supplied to said electrical stimulator; and
an input block that is capable of communicating with said control block and inputs said user's respiratory cycle information acquired in advance to said control block;
wherein said control block is capable of adjusting a first period during which said current is supplied to said electrical stimulator and a second period during which the supply of said current to said electrical stimulator is stopped based on said respiratory cycle information.

2. The respiratory stabilization device of claim 1, wherein said respiratory cycle information includes an exhalation period during which said user exhales air and an inhalation period during which said user inhales air, and the length of said first period is the same as the length of said exhalation period, and the length of said second period is the same as the length of said inhalation period.

3. The respiratory stabilization device of claim 1, wherein said input block has a breath sound sensor that acquires breath sounds of said user and derives said respiratory cycle information based on said breath sounds.

4. The respiratory stabilization device of claim 1, wherein said input block has a flow rate sensor that acquires an air flow rate due to breathing of said user and derives said respiratory cycle information based on said air flow rate.

5. The respiratory stabilization device of claim 1, wherein said input block has a displacement sensor that acquires a displacement of said user's abdomen and derives said respiratory cycle information based on said displacement of said abdomen.

6. A respiratory stabilization device of claim 1, further comprising a stimulation interrupt unit that transmits a stop signal to said control block by an operation of said user, wherein said control block stops supplying said current to said electrical stimulator when it receives said stop signal.

7. A respiratory stabilization device of claim 1, further comprising a display unit that displays instruction images visible to said user, wherein said instruction images including a first instruction image that is displayed during a first period and instructs said user to exhale air, and a second instruction image that is displayed during a second period and instructs said user to inhale air.

8. A respiratory stabilization device in claim 1, further comprising a sound unit that generates instruction sounds audible to said user, wherein said instruction sounds including a first instruction sound that is generated during a first period and instructs said user to exhale air, and a second instruction sound that is generated during a second period and instructs said user to inhale air.

9. The respiratory stabilization device in claim 1, wherein said electrical stimulator is attached to at least the rectus abdominis muscle of said user.

10. A respiratory stabilization device in claim 1, wherein said control block is capable of adjusting a current intensity supplied to said electrical stimulator.

11. A radiation delivery system, comprising:
a respiratory stabilization device, comprising:
an electrical stimulator attached to an abdomen of a user and applying electrical stimulation to said abdomen;
a control block that controls a current supplied to said electrical stimulator; and
an input block that is capable of communicating with said control block and inputs said user's respiratory cycle information acquired in advance to the control unit;
wherein the control block is capable of adjusting a first period during which said current is supplied to said electrical stimulator and a second period during which the supply of said current to said electrical stimulator is stopped based on said respiratory cycle information; and
a radiation delivery unit that delivers radiation to a lesion of a chest or a lesion of an abdomen of said user;
wherein said radiation delivery unit is capable of adjusting at least one of a delivery direction of said radiation or a delivery time of said radiation based on said respiratory cycle information.

12. A radiation delivery system, comprising:
a respiratory stabilization device, comprising:
an electrical stimulator attached to the abdomen of a user and applying electrical stimulation to said abdomen;
a control block that controls a current supplied to said electrical stimulator; and
an input block that is capable of communicating with said control block and inputs said user's respiratory cycle information acquired in advance to said control unit;
wherein said control block is capable of adjusting a first period during which said current is supplied to said electrical stimulator and a second period during which the supply of said current to said electrical stimulator is stopped based on said respiratory cycle information;
a radiation delivery unit that delivers radiation to a lesion in a chest or a lesion in an abdomen of said user; and
a measurement block that measures a respiratory cycle of said user;
wherein said control block in said respiratory stabilization device is capable of adjusting a first period and a second period based on said respiratory cycle, and said radiation delivery unit is capable of adjusting at least one of a delivery direction or a delivery time during which said radiation is delivered based on said respiratory cycle measured by said measurement block.

13. A respiratory stabilization method using a respiratory stabilization device, comprising:
an electrical stimulator attached to a user's abdomen and providing electrical stimulation to the abdomen;
a control block controlling a current supplied to said electrical stimulator, and
an input block capable of communicating with said control block;
wherein said respiratory stabilization method, comprising:
an input step in which respiratory cycle information representing a user's respiratory cycle, which has been previously acquired, is sent to said control block from said input block;
a current supply step in which said control block supplies said current to said electrical stimulator;
wherein said control block is capable of adjusting a first period during which said current is supplied to said electrical stimulator and a second period during which said current supply to said electrical stimulator is stopped based on said respiratory cycle information.
